(19) United States
(12) Patent Application Publication
(10) Pub. No.: US 2024/0423747 A1
(43) Pub. Date: Dec. 26, 2024

(54) DEVICE FOR CONTAINING EXHALED DROPLETS AND AEROSOLS

(71) Applicant: Mary Jane Purcell, Williamswood (CA)

(72) Inventor: Mary Jane Purcell, Williamswood (CA)

(21) Appl. No.: 18/373,815

(22) Filed: Sep. 27, 2023

(30) Foreign Application Priority Data
Jun. 23, 2023 (CA) 3204696

(51) Int. Cl. A61B 90/00; A61G 10/00; A61M 16/00

(52) U.S. Cl. CPC A61B 90/05; A61G 10/005; A61M 16/0093; A61M 2230/432

(57) ABSTRACT

A device for containing droplets and aerosols emitted from a patient's airways is provided. The device comprises a protective shield for being placed at least in front of the patient. At least an access opening is disposed in the protective shield for enabling access therethrough to the patient. At least a cover is mounted to the protective shield such that each of the at least a cover is associated with a respective access opening. Each of the at least a cover is movable between a closed position and an open position. A suction device is in fluid communication with a space behind the protective shield. The suction device substantially removes the droplets and aerosols emitted from the patient's airways.

### FIELD OF THE INVENTION

The present invention relates to devices for containing pathogens, and more particularly to a device for containing droplets and aerosols emitted from a patient's airways.

### BACKGROUND OF THE INVENTION

Various medical procedures such as, for example, spirometry breathing tests to determine if a person has Chronic Obstructive Pulmonary Disease (COPD), Asthma, or Pulmonary Fibrosis, require medical practitioners to interact with a patient's airways which typically generate droplets and aerosols emitted from the patient's airways, potentially exposing the medical practitioner to a high load of dangerous and contagious pathogens such as, for example, COVID-19, contained therein. For example, during the spirometry breathing test the patient is required to remove the mask and cough hard. During coughing large amounts of droplets and aerosols are emitted with the aerosols forming a plume of very small nuclei of 0.3 microns or less which can stay suspended in the air for an extended period of time.

Currently, portable spirometry is typically performed with Personal Protective Equipment (PPE) without additional protection, for example, at bedside in clinical settings or in small, multi-use office spaces which are difficult and time-consuming to clean, thus creating a substantial risk of infection for the medical practitioner as well as for other people entering the room after the spirometry is finished. In facilities where spirometry is considered an Aerosol Generating Maneuver (AGM), or if a patient is considered to be at risk of having Covid-19, costly disposable N-95 masks are used, as well as additional time-consuming infection control cleaning procedures.

In larger hospitals the spirometry test is carried out in a Pulmonary Function Test (PFT) lab with a body plethysmography stationary booth which typically requires approximately one hour preparation time between patients for infection control cleaning due to the aerosols generated during the test.

It is desirable to provide a device for substantially containing droplets and aerosols emitted from a patient's airways.

It is also desirable to provide a device for substantially containing droplets and aerosols emitted from a patient's airways that forms a protective shield between a medical practitioner and the patient's upper body and comprises means for substantially removing the droplets and aerosols emitted from a patient's airways.

It is also desirable to provide a device for substantially containing droplets and aerosols emitted from a patient's airways that forms a protective shield between a medical practitioner and the patient's upper body and comprises means for substantially removing the droplets and aerosols emitted from a patient's airways and means for substantially capturing the removed droplets and aerosols.

It is also desirable to provide a device for substantially containing droplets and aerosols emitted from a patient's airways that forms a protective shield between a medical practitioner and the patient's upper body and comprises means for substantially removing the droplets and aerosols emitted from a patient's airways and means for indicating if the droplets and aerosols have been sufficiently removed.

It is also desirable to provide a device for substantially containing droplets and aerosols emitted from a patient's airways that forms a protective shield between a medical practitioner and the patient's upper body that is easy to move and quick to clean.

### SUMMARY OF THE INVENTION

Accordingly, one object of the present invention is to provide a device for substantially containing droplets and aerosols emitted from a patient's airways.

Another object of the present invention is to provide a device for substantially containing droplets and aerosols emitted from a patient's airways that forms a protective shield between a medical practitioner and the patient's upper body and comprises means for substantially removing the droplets and aerosols emitted from a patient's airways.

Another object of the present invention is to provide a device for substantially containing droplets and aerosols emitted from a patient's airways that forms a protective shield between a medical practitioner and the patient's upper body and comprises means for substantially removing the droplets and aerosols emitted from a patient's airways and means for substantially capturing the removed droplets and aerosols.

Another object of the present invention is to provide a device for substantially containing droplets and aerosols emitted from a patient's airways that forms a protective shield between a medical practitioner and the patient's upper body and comprises means for substantially removing the droplets and aerosols emitted from a patient's airways and means for indicating if the droplets and aerosols have been sufficiently removed.

Another object of the present invention is to provide a device for substantially containing droplets and aerosols emitted from a patient's airways that forms a protective shield between a medical practitioner and the patient's upper body that is easy to move and quick to clean.

According to one aspect of the present invention, there is provided a device for containing droplets and aerosols emitted from a patient's airways. The device comprises a protective shield for being placed at least in front of the patient. At least an access opening is disposed in the protective shield for enabling access therethrough to the patient. At least a cover is mounted to the protective shield such that each of the at least a cover is associated with a respective access opening. Each of the at least a cover is movable between a closed position and an open position. A suction device is in fluid communication with a space behind the protective shield. The suction device substantially removes the droplets and aerosols emitted from the patient's airways.

According to the aspect of the present invention, there is provided a device for containing droplets and aerosols emitted from a patient's airways. The device comprises a protective shield for being placed at least in front of the patient. At least an access opening is disposed in the protective shield for enabling access therethrough to the patient. At least a cover is mounted to the protective shield such that each of the at least a cover is associated with a respective access opening. Each of the at least a cover is movable between a closed position and an open position. A suction device is in fluid communication with a space behind the protective shield. The suction device substantially removes the droplets and aerosols emitted from the patient's airways. The suction device comprises a suction fan with a suction port thereof being connected to a suction conduit which is connected to a suction port disposed in the protective shield.

According to the aspect of the present invention, there is provided a device for containing droplets and aerosols emitted from a patient's airways. The device comprises a protective shield for being placed at least in front of the patient. At least an access opening is disposed in the protective shield for enabling access therethrough to the patient. At least a cover is mounted to the protective shield such that each of the at least a cover is associated with a respective access opening. Each of the at least a cover is movable between a closed position and an open position. A suction device is in fluid communication with a space behind the protective shield. The suction device substantially removes the droplets and aerosols emitted from the patient's airways. The suction device comprises a suction fan with a suction port thereof being connected to a suction conduit which is connected to a suction port disposed in the protective shield. The suction port is disposed in a portion of the protective shield opposite a front of the patient in close proximity to the patient's mouth.

According to the aspect of the present invention, there is provided a device for containing droplets and aerosols emitted from a patient's airways. The device comprises a protective shield for being placed at least in front of the patient. At least an access opening is disposed in the protective shield for enabling access therethrough to the patient. At least a cover is mounted to the protective shield such that each of the at least a cover is associated with a respective access opening. Each of the at least a cover is movable between a closed position and an open position. A suction device is in fluid communication with a space behind the protective shield. The suction device substantially removes the droplets and aerosols emitted from the patient's airways. The suction device comprises a suction fan with a suction port thereof being connected to a suction conduit which is connected to a suction port disposed in the protective shield. A CO₂ sensor is disposed behind the protective shield for sensing a CO₂ concentration and providing CO₂ concentration data in dependence thereon. The CO₂ sensor is disposed between the patient and the protective shield. A display is connected to the CO₂ sensor for displaying the CO₂ concentration data.

According to the aspect of the present invention, there is provided a device for containing droplets and aerosols emitted from a patient's airways. The device comprises a protective shield for being placed at least in front of the patient. At least an access opening is disposed in the protective shield for enabling access therethrough to the patient. At least a cover is mounted to the protective shield such that each of the at least a cover is associated with a respective access opening. Each of the at least a cover is movable between a closed position and an open position. A suction device is in fluid communication with a space behind the protective shield. The suction device substantially removes the droplets and aerosols emitted from the patient's airways. The suction device comprises a suction fan with a suction port thereof being connected to a suction conduit which is connected to a suction port disposed in the protective shield. The suction fan is adapted for providing different suction strength. A CO₂ sensor is disposed behind the protective shield for sensing a CO₂ concentration and providing CO₂ concentration data in dependence thereon. The CO₂ sensor is disposed between the patient and the protective shield. A processor is connected to the CO₂ sensor and the suction fan. The processor controls the suction strength in dependence upon the CO₂ concentration data.

According to the aspect of the present invention, there is provided a device for containing droplets and aerosols emitted from a patient's airways. The device comprises a protective shield for being placed at least in front of the patient. At least an access opening is disposed in the protective shield for enabling access therethrough to the patient. At least a cover is mounted to the protective shield such that each of the at least a cover is associated with a respective access opening. Each of the at least a cover is movable between a closed position and an open position. A suction device is in fluid communication with a space behind the protective shield. The suction device substantially removes the droplets and aerosols emitted from the patient's airways. The protective shield comprises a housing forming an inside space adapted for containing at least a portion of the patient's body therein. The housing is placed on a table-like base with a substantially flat table-top plate. The base has wheels for enabling rolling movement of the device. The table-top plate comprises a cut-out adapted for accommodating the patient's body therein. The housing comprises a front wall, a left-hand side wall, a right-hand side wall, and a top wall forming the inside space for containing the upper body of the patient therein. The suction device is in fluid communication with the inside space through a suction port disposed in the front wall in close proximity to the patient's mouth.

According to the aspect of the present invention, there is provided a method for substantially protecting a medical practitioner from droplets and aerosols emitted from a patient's airways. A device for containing droplets and aerosols emitted from the patient's airways is provided. The device comprises a protective shield for being placed at least in front of the patient. At least an access opening is disposed in the protective shield for enabling access therethrough to the patient. At least a cover is mounted to the protective shield such that each of the at least a cover is associated with a respective access opening. Each of the at least a cover is movable between a closed position and an open position. A suction device is in fluid communication with a space behind the protective shield. The suction device substantially removes the droplets and aerosols emitted from the patient's airways. At least an upper portion of the patient's body is placed behind the protective shield. Using the suction device the droplets and aerosols emitted from the patient's airways are substantially removed.

According to the aspect of the present invention, there is provided a method for substantially protecting a medical practitioner from droplets and aerosols emitted from a patient's airways. A device for containing droplets and aerosols emitted from the patient's airways is provided. The device comprises a protective shield for being placed at least in front of the patient. At least an access opening is disposed in the protective shield for enabling access therethrough to the patient. At least a cover is mounted to the protective shield such that each of the at least a cover is associated with a respective access opening. Each of the at least a cover is movable between a closed position and an open position. A suction device is in fluid communication with a space behind the protective shield. The suction device substantially removes the droplets and aerosols emitted from the patient's airways. At least an upper portion of the patient's body is placed behind the protective shield. Using the suction device the droplets and aerosols emitted from the patient's airways are substantially removed. A CO₂ sensor is disposed behind the protective shield. The CO₂ sensor senses a CO₂ concentration and provides CO₂ concentration data in dependence thereon. The medical practitioner accesses the patient through the at least an access opening if the CO₂ concentration data are indicative of the CO₂ concentration behind the protective shield being below a predetermined threshold. The medical practitioner or a person other than the patient accesses the space behind the protective shield after the patient has left if the CO₂ concentration data are indicative of the CO₂ concentration behind the protective shield are below a predetermined threshold.

According to the aspect of the present invention, there is provided a method for substantially protecting a medical practitioner from droplets and aerosols emitted from a patient's airways. A device for containing droplets and aerosols emitted from the patient's airways is provided. The device comprises a protective shield for being placed at least in front of the patient. At least an access opening is disposed in the protective shield for enabling access therethrough to the patient. At least a cover is mounted to the protective shield such that each of the at least a cover is associated with a respective access opening. Each of the at least a cover is movable between a closed position and an open position. A suction device is in fluid communication with a space behind the protective shield. The suction device substantially removes the droplets and aerosols emitted from the patient's airways. At least an upper portion of the patient's body is placed behind the protective shield. Using the suction device the droplets and aerosols emitted from the patient's airways are substantially removed. A CO₂ sensor is disposed behind the protective shield. The CO₂ sensor senses a CO₂ concentration and provides CO₂ concentration data in dependence thereon. The suction strength of the suction device is adjusted in dependence upon the CO₂ concentration data.

The advantage of the present invention is that it provides a device for substantially containing droplets and aerosols emitted from a patient's airways.

A further advantage of the present invention is that it provides a device for substantially containing droplets and aerosols emitted from a patient's airways that forms a protective shield between a medical practitioner and the patient's upper body and comprises means for substantially removing the droplets and aerosols emitted from a patient's airways.

A further advantage of the present invention is that it provides a device for substantially containing droplets and aerosols emitted from a patient's airways that forms a protective shield between a medical practitioner and the patient's upper body and comprises means for substantially removing the droplets and aerosols emitted from a patient's airways and means for substantially capturing the removed droplets and aerosols.

A further advantage of the present invention is that it provides a device for substantially containing droplets and aerosols emitted from a patient's airways that forms a protective shield between a medical practitioner and the patient's upper body and comprises means for substantially removing the droplets and aerosols emitted from a patient's airways and means for indicating if the droplets and aerosols have been sufficiently removed.

A further advantage of the present invention is that it provides a device for substantially containing droplets and aerosols emitted from a patient's airways that forms a protective shield between a medical practitioner and the patient's upper body that is easy to move and quick to clean.

### BRIEF DESCRIPTION OF THE DRAWINGS

A preferred embodiment of the present invention is described below with reference to the accompanying drawings, in which:

FIG. 1 is a simplified block diagram illustrating in a side view a device for containing droplets and aerosols emitted from a patient's airways according to a preferred embodiment of the invention with the suction device not shown for simplicity;

FIGS. 2 and 3 are simplified block diagrams illustrating in side views movement of droplets and aerosols in the device for containing droplets and aerosols emitted from a patient's airways according to the preferred embodiment of the invention without and with provision of suction, respectively;

FIGS. 4 to 7 are simplified block diagrams illustrating in a perspective front view, a front view, perspective front view, and a side view, respectively, the device for containing droplets and aerosols emitted from a patient's airways according to a preferred embodiment of the invention;

FIG. 8 is a simplified block diagram illustrating in a front view a different placement of the suction port of the device for containing droplets and aerosols emitted from a patient's airways according to a preferred embodiment of the invention;

FIG. 9 is a simplified block diagram illustrating in a top perspective view components of the suction device of the device for containing droplets and aerosols emitted from a patient's airways according to a preferred embodiment of the invention;

FIG. 10 is a simplified block diagram illustrating in a top perspective view a connecting plate of the device for containing droplets and aerosols emitted from a patient's airways according to a preferred embodiment of the invention;

FIG. 11 is a simplified block diagram illustrating control circuitry of the device for containing droplets and aerosols emitted from a patient's airways according to a preferred embodiment of the invention; and,

FIG. 12 is a simplified block diagram illustrating in a side view a device for containing droplets and aerosols emitted from a patient's airways according to another preferred embodiment of the invention.

### DESCRIPTION OF THE PREFERRED EMBODIMENT

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described.

While the description of the preferred embodiments hereinbelow is with reference to a device for containing droplets and aerosols emitted from a patient's airways during spirometry breathing tests, it will become evident to those skilled in the art that the embodiments of the invention are not limited thereto, but are also adaptable for substantially protecting medical practitioners from droplets and aerosols emitted from a patient's airways during various other medical interventions such as, for example, for swab testing, administering vaccines, or drawing blood.

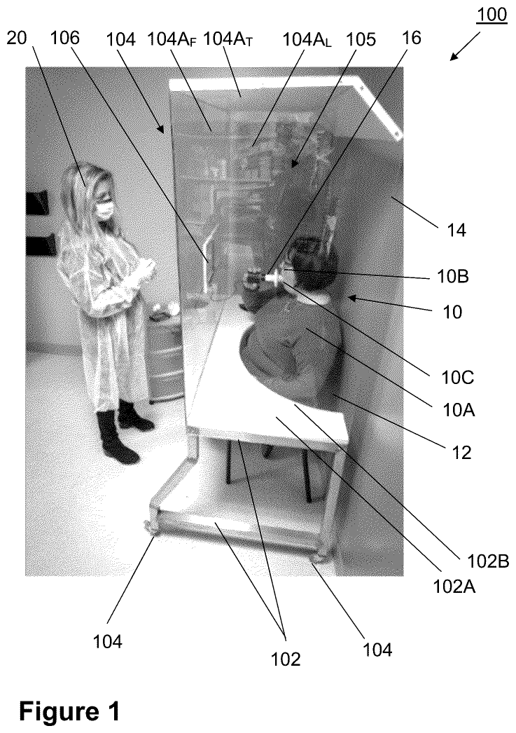

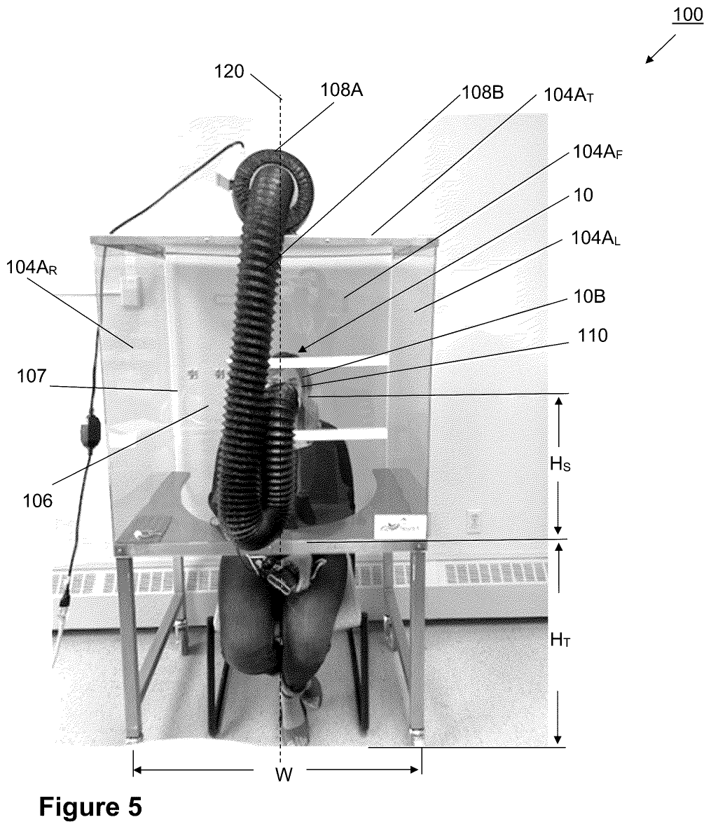

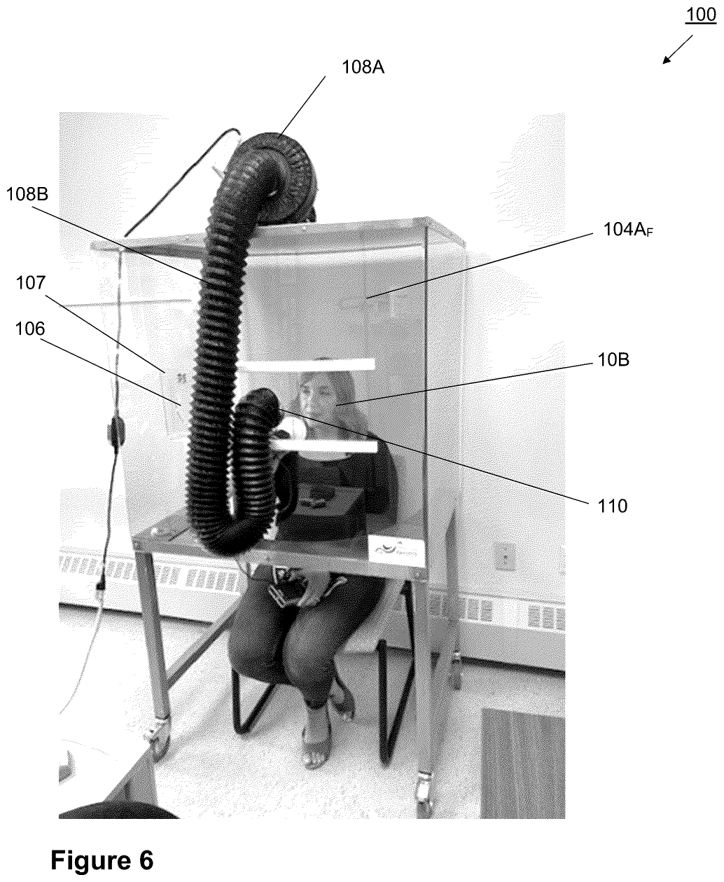

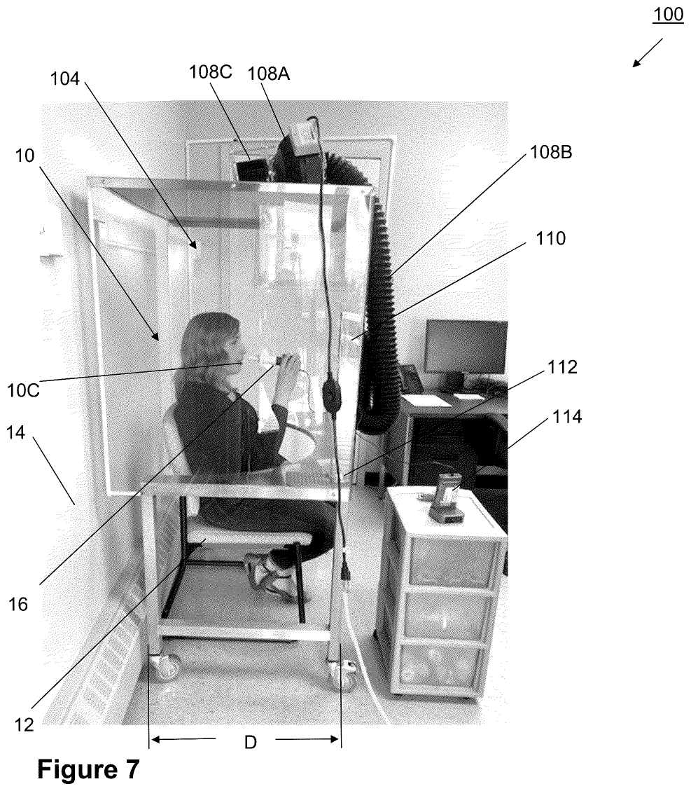

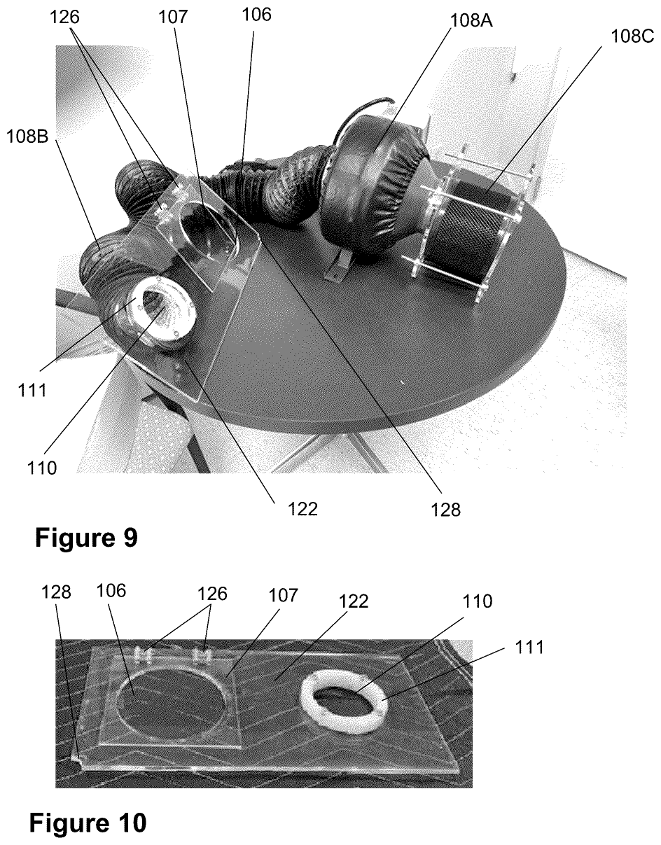

Referring to FIGS. 1 to 11 a device 100 for containing droplets and aerosols emitted from a patient's airways according to a preferred embodiment of the invention is provided. The device 100 comprises a protective shield for being placed at least in front of the patient, thus providing a barrier between the patient 10 and a medical practitioner 20 performing a medical intervention such as a spirometry breathing test on the patient 10. Preferably, the protective shield comprises a housing 104 that forms an inside space 105 adapted for containing at least the upper body 10A of the patient 10 therein. For example, the housing comprises front wall 104A_{F}, left-hand side wall 104A_{L}, right-hand side wall 104A_{R}, and top wall 104A_{T} forming the inside space 105 for containing the upper body 10A of the patient 10 therein. Further preferably, the housing 104 is placed on a, for example, table-like base 102 having wheel assemblies 104 such as, for example, swivel casters mounted thereto for enabling rolling movement of the device 100 in order to easily move the device 100 within a building. The base has, for example, a substantially flat table-top plate 102A comprising a C-shaped cut-out 102B for accommodating the upper body 10A of a patient 10 seated on a chair 12 or wheelchair therein, as illustrated in FIG. 1.

Access opening 106 is disposed in the housing 104 for enabling access therethrough to the patient, for example, to enable the medical practitioner to access the spirometer's mouthpiece 16 and filter, to make sure the patient 10 has the mouthpiece 16 securely in place, or to hand a puffer to the patient 10 if necessary. The access opening 106 may be provided having various shapes such as, for example, circular, square, or rectangular, and sizes depending on design preferences. Preferably, the access opening 106 is disposed in the front wall 104A_{F} opposite the patient's face 10B and in proximity to the patient's mouth 10C to enable easy access to the patient's mouth while handling the spirometer's mouthpiece 16. Cover 107 is movable between a closed position, to protect the medical practitioner from droplets and aerosols emitted from the patient's airways during the spirometry breathing test, and an open position, to enable access to the patient 10. The cover 107 is, for example, pivotally or slidably movable mounted to the housing 104, thus enabling opening by flipping the same upward or sliding it sideways. Alternatively, instead of a rigid cover a flexible membrane type cover may be employed. As is evident to a person skilled in the art, the access opening 106 may be placed at another location as well as more than one access opening 106 with a respective cover 107 may be provided, depending on design preferences.

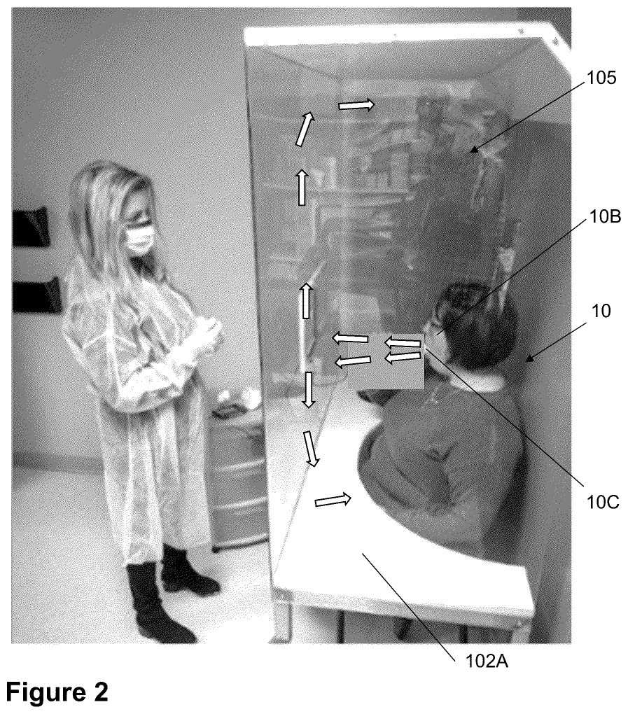

FIG. 2 illustrates movement of droplets and aerosols emitted from the patient's airways during the spirometry breathing test, for example, when the patient 10 is asked to cough, inside the housing 104. As indicated by the block arrows, the droplets and aerosols after being emitted from the patient's mouth 10C are propelled towards the front wall 104A_{F} opposite the patient's face 10B. While larger droplets impact the front wall 104A_{F} or fall onto the table-top plate 102A, smaller droplets and aerosols are first deflected upward, downward, and sideways (not shown) and then in proximity to the top wall 104A_{T} or the table-top plate 102A are deflected towards a backward direction, resulting in a distribution of, particularly, the aerosols which can stay suspended in air for a substantial period of time, within the inside space 105 of the housing 104.

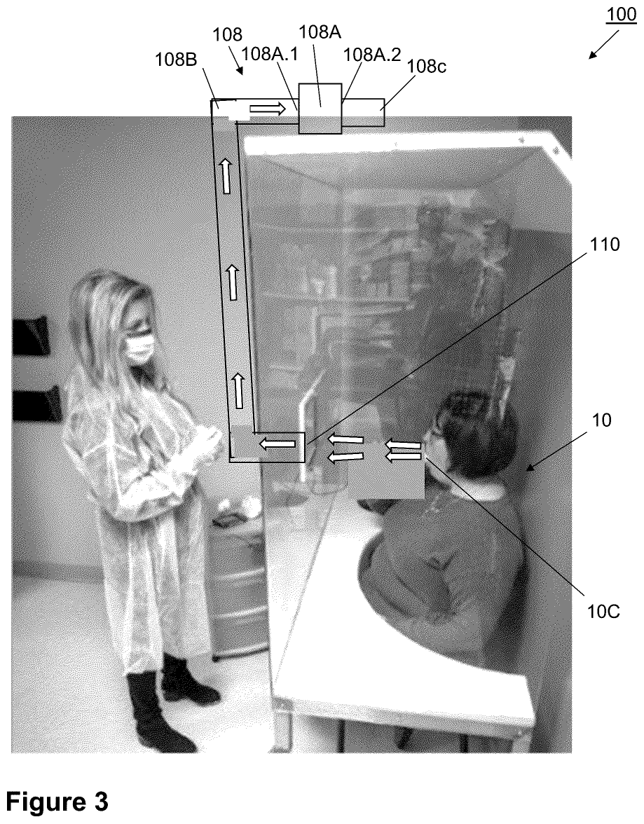

Providing a suction device 108 in fluid communication with the inside space 105, as illustrated in FIG. 3, enables substantial removal of the droplets and aerosols emitted from the patient's mouth 10C. The suction device comprises a suction fan 108A with a suction port 108A. 1 thereof being connected to a suction conduit 108B such as, for example, a flexible suction hose, which is connected to suction port 110 disposed, preferably, in the front wall 104A_{F} of the housing 104 in close proximity to the patient's mouth 10C. Alternatively, rigid tubing may be employed for connecting the suction fan 108A to the suction port 110. Further preferably, the suction device 108 comprises a filter 108C such as, for example, a High Efficiency Particulate Absorbing (HEPA) filter, connected to an exhaust port 108A.2 of the suction fan 108A for substantially capturing the removed droplets and aerosols. Alternatively, the filter 108C may be omitted, for example, when it is possible to connect the exhaust port 108A.2 of the suction fan 108A to the outside using a hose or a pipe. As indicated by the block arrows, the droplets and aerosols after being emitted from the patient's mouth 10C are propelled towards the front wall 104A_{F} opposite the patient's face 10B and directed through the suction provided by the suction fan 10A towards the suction port 110 and transmitted via the suction conduit 108B and the suction fan 108A to the filter 108C for capture therein. With the suction port 110 being placed sufficiently close to the patient's mouth 10C and sufficient suction provided by the suction fan 108A the suction device 108 is capable of substantially removing the droplets and aerosols before being distributed within the housing 104.

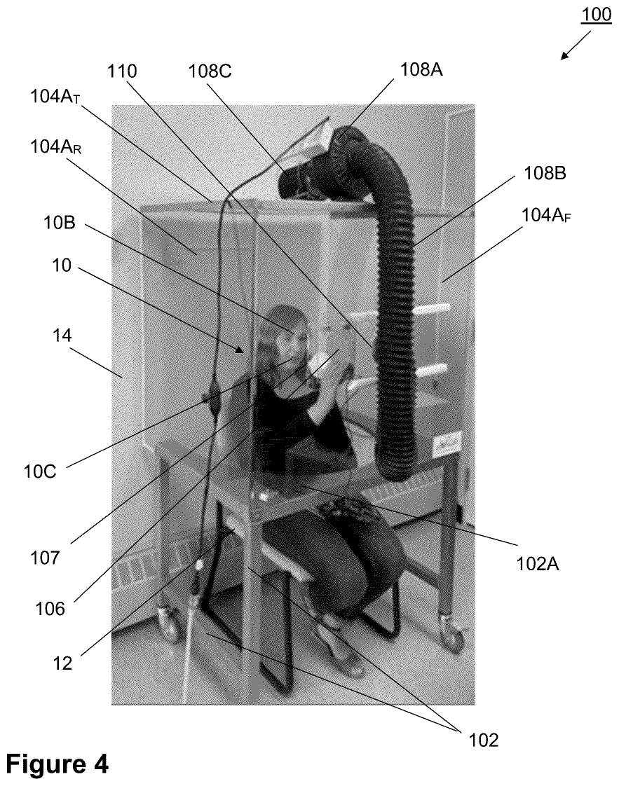

Preferably, as illustrated in FIGS. 4 to 7, the suction port 110 is placed on central line 120 of the housing 104 at a height H_{S} above the table-top plate 102A of approximately 15 inches, which substantially coincides with a location opposite the patient's mouth 10C when the patient 10 is seated on chair 12 inside the cut-out 102B. With the suction port 110 placed at a centre location substantially opposite the patient's mouth 10C, the droplets and aerosols emitted from the patient's mouth 10C are propelled substantially directly towards the suction port 110. The placement of the suction port 110 at the centre location requires the access opening 106 to be placed off-centre adjacent to the suction port 106.

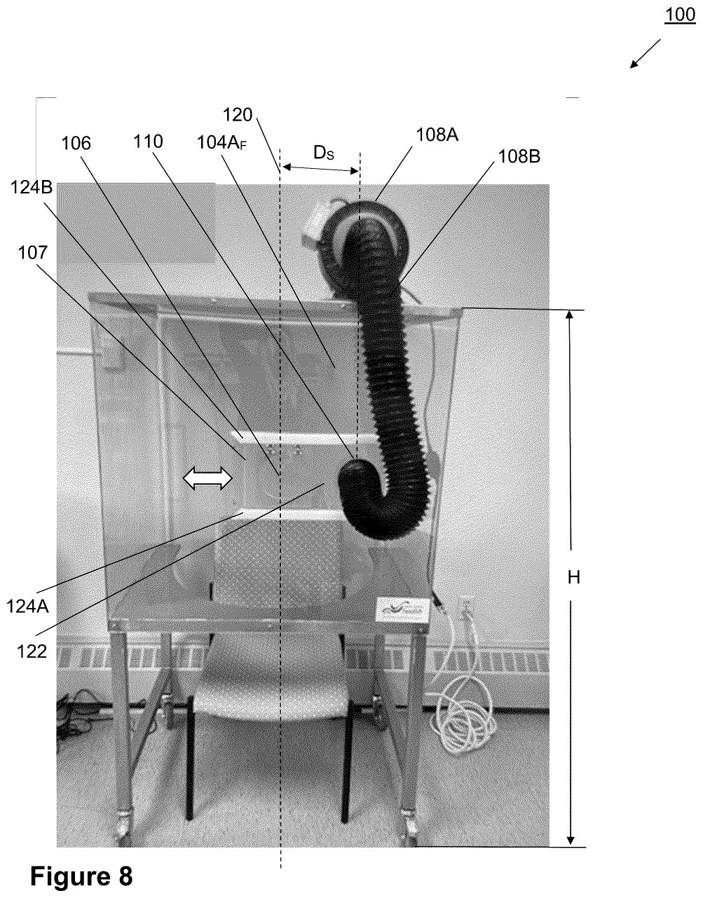

Alternatively, if it is desired or necessary for performing the medical intervention, the access opening 106 is placed at a centre location with the suction port 110 being placed adjacent thereto at a close distance D_{S}, as illustrated in FIG. 8, which still enables direct substantial removal of the droplets and aerosols emitted from the patient's mouth 10C by directing the same towards the suction port 110 through the suction provided by the suction fan 10A.

Further alternatively, the suction port 110 may be placed at locations such as, for example, in a bottom portion of the front wall 104A_{F} of the housing 104 or in a front portion of the table-top plate 102A in proximity to the centre 120. Of course, the suction port 110 may also be placed at other locations at a larger distance to the front centre at the cost of reduced efficiency and/or increased suction required depending on design preferences.

Further alternatively, the suction fan 108A and the filter 108C may be placed at a different location than on top of the housing 104, as illustrated in the FIGS. 3 to 8, such as, for example, mounted to the base 102. Provision of the suction fan 108A and the filter 108C connected to a sufficiently long flexible hose 108B may also enable the medical practitioner to place the suction fan 108A and the filter 108C at a location of his/her choosing such as, for example, the top of the housing 104, the base 102, the floor, or a piece of furniture.

Referring to FIGS. 8 to 10, the access opening 106 and the suction port 110 are, preferably, mounted to connecting plate 122 which is easily removable/re-attachable mounted to the front wall 104A_{F} of the housing 104, for example, by providing guiding rails 124A, 124B fixedly mounted to the front wall 104A_{F} for holding the connecting plate 122 as well as for guiding the connecting plate 122 while sliding the same therebetween for removal/re-attachment, as indicated by the block arrow in FIG. 8. The connecting plate 122 has mounted thereto a ring or flange 111 surrounding the suction port 110 for enabling mounting of the suction hose 108B thereto using, for example, a hose clamp. The access opening 106 is covered by cover plate 107 pivotally movable mounted to the connecting plate 122 via hinges 126. Alternatively, the cover plate 107 may be slidably movable mounted to the connecting plate 122 or be provided as a flexible membrane type cover. Optionally, a small notch 128 is disposed in the connecting plate 122 or, alternatively, in the cover plate 107, to enable passing of an equipment cable therethrough while the access opening 106 is covered.

Provision of the easily removable/re-attachable connecting plate 122 substantially facilitates cleaning of the suction port 110 and the access opening 106 with the cover plate 107 as part of the complete cleaning of the housing 104 between patient's. Furthermore, the easily removable/re-attachable connecting plate 122 also enables switching of the location of the access opening 106 and the suction port 110 by simply replacing the connecting plate 122 having the access opening 106 at the centre location, as illustrated in FIGS. 8 to 10, with a connecting plate 122 having the suction port 110 at the centre location.

In an example implementation, the housing 104 is made of, for example, ¼ inch thick clear polycarbonate sheet material, the base 102 is made of stainless steel profiles and the table-top plate 102A is made of stainless steel or High Density Polyethylene (HDPE). The device 100 has a total height H of 76 inches, a width W of 48 inches, a depth D of 24 inches, and the table-top plate 102A is placed at a height H_{T} of 30 inches. The dimensions of the device 100 have been chosen to enable comfortable seating of the patient inside the housing 104 while ensuring ergonomically positioning of the access opening 106 to prevent poor body posture and possibly body discomfort of medical practitioner. Furthermore, the materials and dimensions of the device 100 have been chosen to enable easy cleaning and disinfecting of the same between patients with easy cleanable surfaces that can easily be accessed and reached during cleaning, thus wait times between patients is substantially reduced.

The suction device 108 comprises a commercially available suction fan 108A (VIVOSUN® IN-LINE DUCT FAN) having a three speed variable suction strength with a maximum suction strength of 200 CFM. The suction port 108A. 1 of the suction fan 108A is connected to the suction port 110 via a commercially available flexible hose having a four inch diameter. The exhaust port 108A.2 of the suction fan 108A is connected to a commercially available HEPA filter (DonLeeving True HEPA filter).

As is evident to a person skilled in the art, the device 100 is not limited thereto but the shape, the dimensions, and the materials of the base 102 and the housing 104, as well as the suction fan 108A, the conduit 108B, and the filter 108C, may be varied depending on design preferences.

Further preferably, the device 100 comprises a CO₂ sensor 112 such as, for example, a commercially available NonDispersive InfraRed (NDIR) CO₂ sensor, disposed in the housing 104 for sensing a CO₂ concentration and providing CO₂ concentration data in dependence thereon. Further preferably, the CO₂ sensor is disposed between the patient 10 and the front wall 104A_{F} of the housing 104, for example, on the table-top plate 102A in proximity to the front wall 104A_{F} or mounted to the front wall 104A_{F} in proximity to a location opposite the patient's mouth 10C. The CO₂ sensor 112 is connected, for example, to a display 114 placed outside the housing 104 for displaying the CO₂ concentration data. The CO₂ concentration is an indicator of air emitted from the patient's airways and, therefore, also an indicator of the concentration droplets and aerosols emitted from the patient's airways together with the air. For example, without a person placed inside the housing 104 the CO₂ concentration is 400 ppm and once a patient 10 is seated inside the housing 104 with the face mask taken off and no suction provided the CO₂ concentration rises to about 1700 ppm.

In operation, the patient is asked to sit on a chair 12 which is, for example, placed in front of an easily cleanable wall 14. The device 100 is then moved towards the patient 10 seated on the chair 12 such that the patient's upper body 10A is placed with the inside space 105 of the housing 104, as illustrated in FIGS. 1 to 7. Once the patient 10 is placed within the inside space 105 of the housing 104 and has removed the face mask, the suction fan 108A is turned on for removing the droplets and aerosols emitted from the patient's airways. For example, the suction fan 108A is continuously operating on at least LOW speed while the patient 10 is placed inside the housing 104. Prior to opening the cover 107, or when the patient 10 is coughing, or after the medical intervention is finished, the speed of the suction fan 108A is increased to MED or HIGH to provide increased suction strength.

The medical practitioner accesses the patient through the access opening 106 if the CO₂ concentration data are indicative of the CO₂ concentration inside the housing being below a predetermined first threshold. After the patient has left, the medical practitioner or another person accesses the inside space 105 for cleaning and disinfecting the same if the CO₂ concentration data are indicative of the CO₂ concentration inside the housing 104 being below a predetermined second threshold which can be different from the first threshold. For example, the second threshold may be chosen to be lower such as, for example, 400 ppm, than the first threshold such as, for example, 800 ppm, since in the first instance the medical practitioner is exposed to the air emitted by the patient 10 for only a short time through the relatively small access opening 106 while in the second instance the medical practitioner or another person cleaning the housing 104 is placed inside the same and typically spends more time than the medical practitioner when accessing the patient through the access opening 106.

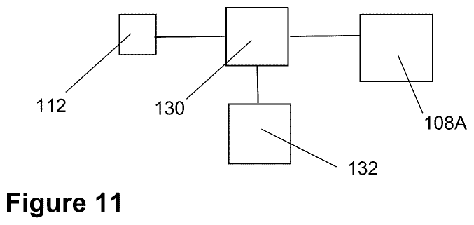

Optionally, as illustrated in FIG. 11, the suction fan 108A and the CO₂ sensor 112 are connected to a processor 130 such as, for example, a commercially available Field Programmable Gate Array (FPGA) for controlling the suction strength provided by the suction fan 108A in dependence upon CO₂ concentration data provided by the CO₂ sensor 112 and user input data such as, for example, CO₂ concentration thresholds, received from user interface 132 such as, for example, a commercially available touchscreen, connected thereto. The suction strength may be continuously adjusted between a LOW and a HIGH level, or in steps such as, for example, LOW, MED, and HIGH, as described hereinabove with respect to the manual operation.

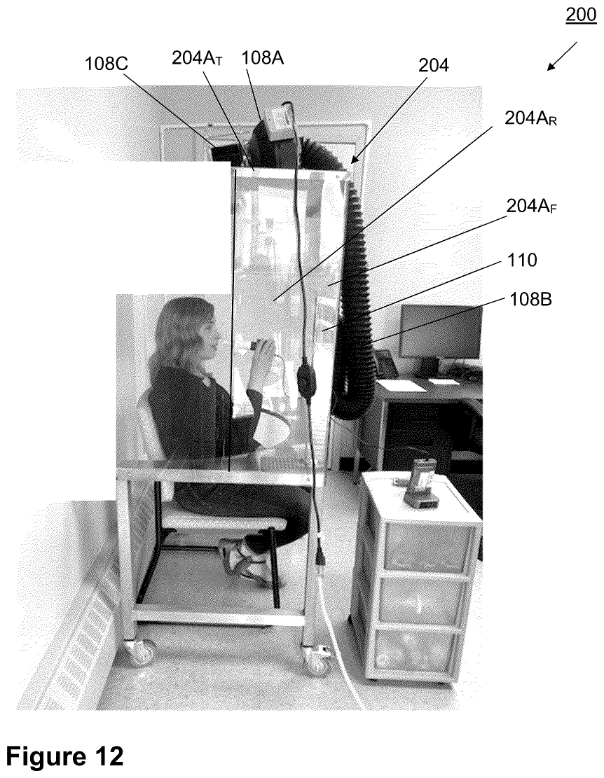

Referring to FIG. 12 a device 200 for containing droplets and aerosols emitted from a patient's airways according to another preferred embodiment of the invention is provided. As is evident to a person skilled in the art, the device 100 is not limited to the shape and size of the housing 104 and base 102 as described hereinabove, but may be varied widely depending on design preferences. For example, provision of sufficient suction via suction fan 108A enables substantially reducing the depth of the left-hand side wall 204A_{L}, the right-hand side wall 204A_{R}, and the top wall 204A_{T} of the housing 204 since most of the droplets and aerosols will be drawn towards the front wall 204A_{F} and, in particular, towards the suction port 110. Of course, the depth of the base 102 and the table-top plate 102A may also be reduced accordingly. It is noted that the device 200 does not need to be placed against a wall 14, since most of the droplets and aerosols will be drawn towards the suction port 110 disposed in the front wall 204A_{F}, but can be placed at any location in a room and the patient can be seated without moving the device 200.

Furthermore, the table-like base 102 may be omitted and the housing 104 may be extended downward and mounted to a base frame in close proximity to the floor. In a further simplification the top wall may be omitted, leaving the front wall and the side walls, or ultimately, only the front wall may be employed as a barrier between the medical practitioner and the patient seated behind the barrier which can still provide protection for some medical interventions such as, for example, swab testing, administering vaccines, or drawing blood, if sufficient suction is provided.

Optionally, the device 100, 200 or further modifications thereof as described hereinabove may be adapted for being placed in a vehicle such as, for example, a van or a bus, to provide medical interventions such as, for example, spirometry breathing tests, to under-served areas.

The present invention has been described herein with regard to preferred embodiments. However, it will be obvious to persons skilled in the art that a number of variations and modifications can be made without departing from the scope of the invention as described herein.

What is claimed is:

1. A device for containing droplets and aerosols emitted from a patient's airways comprising:
a protective shield for being placed at least in front of the patient;
at least an access opening disposed in the protective shield for enabling access therethrough to the patient;
at least a cover mounted to the protective shield such that each of the at least a cover is associated with a respective access opening, each of the at least a cover being movable between a closed position and an open position; and,
a suction device in fluid communication with a space behind the protective shield, the suction device for substantially removing the droplets and aerosols emitted from the patient's airways.

2. The device according to claim 1 wherein the suction device comprises a suction fan with a suction port thereof being connected to a suction conduit and wherein the suction conduit is connected to a suction port disposed in the protective shield.

3. The device according to claim 2 wherein the suction port is disposed in a portion of the protective shield opposite a front of the patient.

4. The device according to claim 3 wherein the suction port is disposed in close proximity to the patient's mouth.

5. The device according to claim 2 wherein the suction device comprises a filter connected to an exhaust port of the suction fan, the filter for substantially capturing the removed droplets and aerosols.

6. The device according to claim 2 wherein the suction fan is adapted for providing different suction strength.

7. The device according to claim 6 comprising a CO₂ sensor disposed behind the protective shield for sensing a CO₂ concentration and providing CO₂ concentration data in dependence thereon.

8. The device according to claim 7 wherein the CO₂ sensor is disposed between the patient and the protective shield.

9. The device according to claim 8 wherein the CO₂ sensor is disposed in proximity to the patient's mouth.

10. The device according to claim 7 comprising a display connected to the CO₂ sensor for displaying the CO₂ concentration data.

11. The device according to claim 7 comprising a processor connected to the CO₂ sensor and the suction fan, the processor for controlling the suction strength in dependence upon the CO₂ concentration data.

12. The device according to claim 1 wherein the protective shield comprises a housing forming an inside space adapted for containing at least a portion of the patient's body therein.

13. The device according to claim 12 comprising a table-like base with a substantially flat table-top plate having the housing placed thereon, the base having wheels for enabling rolling movement of the device.

14. The device according to claim 13 wherein the table-top plate comprises a cut-out adapted for accommodating the patient's body therein.

15. The device according to claim 14 wherein the housing comprises a front wall, a left-hand side wall, a right-hand side wall, and a top wall forming the inside space for containing the upper body of the patient therein and wherein the suction device is in fluid communication with the inside space through a suction port disposed in the front wall in close proximity to the patient's mouth.

16. A method for substantially protecting a medical practitioner from droplets and aerosols emitted from a patient's airways comprising:
providing a device for containing droplets and aerosols emitted from the patient's airways, the device comprising:
a protective shield for being placed at least in front of the patient;
at least an access opening disposed in the protective shield for enabling access therethrough to the patient;
at least a cover mounted to the protective shield such that each of the at least a cover is associated with a respective access opening, each of the at least a cover being movable between a closed position and an open position; and,
a suction device in fluid communication with a space behind the protective shield, the suction device for substantially removing the droplets and aerosols emitted from the patient's airways;
placing at least an upper portion of the patient's body behind the protective shield; and,
using the suction device substantially removing the droplets and aerosols emitted from the patient's airways.

17. The method according to claim 16 comprising:
providing a CO₂ sensor disposed behind the protective shield; and,
sensing a CO₂ concentration and providing CO₂ concentration data in dependence thereon.

18. The method according to claim 17 comprising:
the medical practitioner accessing the patient through the at least an access opening if the CO₂ concentration data are indicative of the CO₂ concentration behind the protective shield being below a predetermined threshold.

19. The method according to claim 17 comprising:
the medical practitioner or a person other than the patient accessing the space behind the protective shield after the patient has left if the CO₂ concentration data are indicative of the CO₂ concentration behind the protective shield being below a predetermined threshold.

20. The method according to claim 19 comprising increasing a suction strength of the suction device after the patient has left.

21. The method according to claim 17 comprising adjusting the suction strength of the suction device in dependence upon the CO₂ concentration data.
